(19) United States
(12) Patent Application Publication
(10) Pub. No.: US 2010/0145099 A1
(43) Pub. Date: Jun. 10, 2010

(54) NOVEL POLYMORPHIC FORMS OF MILNACIPRAN HYDROCHLORIDE

(75) Inventors: Roshan Ramesh Medhane, Nasik (IN); Nitin Maheswari, Delhi (IN); Keshav Deo, Haryana (IN); Mohan Prasad, Haryana (IN); Joydeep Kant, Haryana (IN)

Correspondence Address: Ranbaxy Inc., Intellectual Property Department, 600 College Road East, PRINCETON, NJ, 08540, US

(73) Assignee: RANBAXY LABORATORIES LIMITED, GURAGAON HARYANA (IN)

(21) Appl. No.: 12/528,983

(22) PCT Filed: Feb. 28, 2008
(86) PCT No.: PCT/IB08/50736

§ 371 Date: Feb. 25, 2010

(30) Foreign Application Priority Data
Feb. 28, 2007 (IN) 447/DEL/2007

(51) Int. Cl. C07C 237/00

(52) U.S. Cl. 564/164

(57) ABSTRACT

The present invention relates to polymorphic forms of milnacipran hydrochloride. The polymorphic forms are designated as Form (I), Form (II), Form (III), Form (IV) and Form V of milnacipran hydrochloride. The present invention also relates to processes for the preparation of the polymorphic forms.

### FIELD OF THE INVENTION

The present invention relates to polymorphic forms of milnacipran hydrochloride. The polymorphic forms are designated as Form I, Form II, Form III, Form IV and Form V of milnacipran hydrochloride. The present invention also relates to processes for the preparation of the polymorphic forms.

### BACKGROUND OF THE INVENTION

(1R,2S)-rel-2-(Aminomethyl)-N,N-diethyl-1-phenylcyclopropanecarboxamide hydrochloride is commonly known as milnacipran hydrochloride of Formula I:

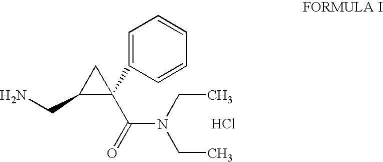

Milnacipran hydrochloride is a Norepinephrine Serotonin Reuptake Inhibitor (NSRI) and it is useful in the treatment of depression and chronic pain conditions like Fibromyalgia and Lupus.

U.S. Pat. No. 4,478,836 provides a process for the preparation of milnacipran hydrochloride by salifying milnacipran base with hydrochloric acid. In this method, milnacipran hydrochloride is obtained as white crystals with a melting point of 180° C. and a characteristic IR peaks at 1620 cm⁻¹. However, the solvents involved in the salification step are not disclosed in this patent.

European Patent No. 0 200 638 B1 provides a process for the preparation of milnacipran hydrochloride by the addition of ethanolic hydrochloric acid to crude milnacipran base. In this method, milnacipran hydrochloride is obtained with a melting point of 180° C. and a characteristic IR peaks at 1610 cm⁻¹.

Japanese Patent No. 2006-008569 A2 provides a process for the preparation of milnacipran hydrochloride. In this method, milnacipran base is treated with hydrogen chloride-ethylacetate followed by the addition of ethyl acetate and isopropyl alcohol, and concentration of the reaction mixture to obtain milnacipran hydrochloride as white powder.

### SUMMARY OF THE INVENTION

The present inventors have prepared polymorphic forms of milnacipran hydrochloride, designated as Form I, Form II, Form III, Form IV and Form V. The present polymorphic forms have characteristic XRPD, FTIR and DSC patterns. The polymorphic forms of the present invention are stable and suitable to prepare pharmaceutical dosage forms.

### DETAILED DESCRIPTION OF THE INVENTION

A first aspect of the present invention provides a polymorphic Form I of milnacipran hydrochloride having substantially the same XRPD pattern as depicted in FIG. 1 of the accompanied drawing. The XRPD of Form I shows characteristic peaks at 2θ values 5.97, 7.75, 11.46, 11.93, 12.48, 13.52, 14.32, 15.52, 16.56, 17.03, 17.63, 18.37, 18.67, 19.26, 19.63, 20.54, 21.11, 21.68, 22.15, 22.96, 24.24, 24.38, 24.58, 25.12, 25.91, 26.69, 26.93, 27.31, 27.98, 28.70, 29.51, 30.25, 30.70, 31.15, 31.84, 32.29, 32.87, 34.13, 34.83 and 35.64±0.2θ. The polymorphic Form I has substantially the same FTIR pattern as depicted in FIG. 2 of the accompanied drawing. The polymorphic Form I has substantially the same DSC thermogram as depicted in FIG. 3 of the accompanied drawing. The DSC thermogram shows one characteristic endothermic peaks between about 177° and about 179° C.

A second aspect of the present invention provides a process for the preparation of the polymorphic Form I of milnacipran hydrochloride, which comprises
a) dissolving milnacipran hydrochloride in a C₁₋₃ alkanol,
b) treating the solution obtained in step a) with an ether solvent, and
c) isolating Form I of milnacipran hydrochloride from the mixture thereof.

Milnacipran hydrochloride is dissolved in a C₁₋₃ alkanol, such as in methanol. The dissolution process may also be accompanied by stirring and/or heating to effect complete dissolution. The solution so obtained is treated with an ether solvent. The ether solvent is employed in this step as an anti-solvent. The ether solvent is selected from the group consisting of diisopropyl ether, diethyl ether, methyl t-butyl ether and di-t-butyl ether. Diisopropyl ether is preferably employed. The treatment with ether solvent is preferably carried out by adding the alcoholic solution of milnacipran hydrochloride to the ether solvent at about 0° C. to about 30° C. The reaction mixture so obtained is stirred to effect complete precipitation of the solid. The solid so obtained is isolated from the mixture by filtration and/or concentration to obtain Form I of milnacipran hydrochloride.

Another aspect of the present invention provides a process for the preparation of the polymorphic Form I of milnacipran hydrochloride, which comprises
a) dissolving milnacipran hydrochloride in a C₁₋₃ alkanol,
b) partially or completely removing the solvent from the solution obtained in step a), and
c) isolating Form I of milnacipran hydrochloride from the mixture thereof.

Milnacipran hydrochloride is dissolved in a C₁₋₃ alkanol, preferably in methanol. The dissolution process may also be accompanied by stirring and/or heating to effect complete dissolution. The solvent is removed from the obtained solution. The solvent is removed partially or completely. The solvent removal may be achieved by distillation under vacuum. The solid so obtained is isolated from the mixture by filtration and/or concentration to obtain Form I of milnacipran hydrochloride.

A further aspect of the present invention provides a process for the preparation of the polymorphic Form I of milnacipran hydrochloride, which comprises
a) dissolving milnacipran hydrochloride in a water miscible ether at a temperature of about 50° C. or more,
b) cooling the solution obtained in step a) to a temperature of about 35° C. or less, and
c) isolating Form I of milnacipran hydrochloride from the mixture thereof.

Milnacipran hydrochloride is treated with a water miscible ether and dissolved by heating the reaction mixture to a temperature of about 50° C. or more. The water miscible ether may be 1,4-dioxane. The solution so obtained is subsequently cooled to a temperature of about 35° C. or less. The solid so obtained is isolated from the mixture by filtration and/or concentration to obtain Form I of milnacipran hydrochloride.

Yet another aspect of the present invention provides a process for the preparation of the polymorphic Form I of milnacipran hydrochloride, which comprises
a) treating milnacipran hydrochloride with an aromatic hydrocarbon solvent,
b) heating the reaction mixture obtained in step a) to a temperature of about 60° C. or more,
c) cooling the reaction mixture obtained in step b) to a temperature of about 35° C. or less, and
d) isolating Form I of milnacipran hydrochloride from the mixture thereof.

Milnacipran hydrochloride is treated with an aromatic hydrocarbon solvent and heated to a temperature of about 60° C. or above. The aromatic hydrocarbon solvent is may be benzene substituted with one or more alkyl groups, such as toluene. The mixture so obtained is subsequently cooled to a temperature of about 35° C. or less. The solid so obtained is isolated from the mixture by filtration and/or concentration to obtain Form I of milnacipran hydrochloride.

The present invention also provides a process for the preparation of the polymorphic Form I of milnacipran hydrochloride, which comprises
a) treating milnacipran hydrochloride with an aliphatic ester solvent and optionally a C₁₋₃ alkanol,
b) treating the mixture obtained in step a) with an acid, and
c) isolating Form I of milnacipran hydrochloride from the mixture thereof.

Milnacipran hydrochloride is treated with an aliphatic ester solvent. The aliphatic ester solvent is preferably ethyl acetate. The reaction mixture may be further treated with a C₁₋₃ alkanol, such as isopropyl alcohol. The mixture is subsequently treated with an acid. The acid may be an alcoholic solution of hydrochloric acid. The mixture so obtained is heated to a temperature of about 60° C. or more and cooled to a temperature of about 20° C. or less. The solid so obtained is isolated from the mixture by filtration and/or concentration to obtain Form I of milnacipran hydrochloride.

Another aspect of the present invention provides polymorphic Form II of milnacipran hydrochloride having substantially the same XRPD pattern as depicted in FIG. 4 of the accompanied drawing. The XRPD of Form II shows characteristic peaks 2θ values 5.95, 11.45, 11.91, 14.32, 18.37, 18.66, 21.11, 21.67, 22.96, 24.23, 24.38, 24.57, 25.39 and 27.67±0.2θ. Form II is further characterized by peaks at 2θ values 5.95, 7.74, 11.45, 11.91, 12.48, 13.51, 14.32, 15.51, 16.53, 17.02, 17.62, 18.37, 18.66, 19.23, 19.63, 20.53, 21.11, 21.67, 22.13, 22.96, 24.23, 24.38, 24.57, 25.12, 25.39, 25.92, 26.68, 26.91, 27.31, 27.67, 27.95, 28.69, 29.50, 30.23, 30.69, 31.14, 31.85, 32.26, 32.88, 34.13, 34.80 and 35.63±0.20. The polymorphic Form II has substantially the same FTIR pattern as depicted in FIG. 5 of the accompanied drawing. The polymorphic Form II has substantially the same DSC thermogram as depicted in FIG. 6 of the accompanied drawing. The DSC thermogram shows one characteristic endothermic peaks between about 175° and about 177° C.

The present invention provide's a process for the preparation of the polymorphic Form II of milnacipran hydrochloride, which comprises
a) dissolving milnacipran hydrochloride in an aqueous solvent,
b) spray drying the solution obtained in step a) in a spray dryer, and
c) collecting Form II of milnacipran hydrochloride from the spray dryer.

Milnacipran hydrochloride is dissolved in an aqueous solvent. The aqueous solvent can be water or a mixture of water with one or more water miscible organic solvents. The dissolution process may also be accompanied by stirring and/or heating to effect complete dissolution. The resultant solution is fed to a spray dryer. The inlet and outlet temperatures, feed rate, and atomizer type are adjusted to optimize output and particle size. The air inlet temperature may be controlled to be in the range from about 60° C. to about 100° C. Compressed air or an inert gas such as nitrogen can be used as a carrier gas for the drying process. After the drying process, milnacipran hydrochloride is collected from the spray dryer and optionally further dried under vacuum to obtain Form II of milnacipran hydrochloride.

Another aspect of the present invention provides a polymorphic Form III of milnacipran hydrochloride having substantially the same XRPD pattern as depicted in FIG. 7 of the accompanied drawing. The XRPD of Form III shows characteristic peaks at 2θ values 5.94, 11.44, 11.90, 14.31, 18.36, 18.65, 21.10, 21.66, 22.95, 24.22, 24.57, 29.70, 31.68, 33.42, 33.93, and 35.39±0.2θ. Form III is further characterized by peaks at 2θ values 5.94, 7.73, 11.44, 11.90, 12.47, 13.50, 14.31, 15.51, 16.53, 17.01, 17.61, 18.36, 18.65, 19.23, 19.62, 20.41, 21.10, 21.66, 22.11, 22.95, 24.22, 24.57, 25.11, 25.90, 26.68, 26.91, 27.31, 27.94, 28.69, 29.70, 30.25, 30.67, 31.14, 31.68, 32.27, 32.87, 33.42, 33.93, 34.12, 34.80, 35.39 and 35.64±0.2θ.

Yet another aspect of the present invention provides a process for the preparation of the polymorphic Form III of milnacipran hydrochloride, which comprises
a) dissolving milnacipran base in a C₁₋₃ alkanol,
b) treating the solution obtained in step a) with hydrochloric acid,
c) treating the solution obtained in step b) with a C₄₋₁₀ alkane, and
d) isolating Form III of milnacipran hydrochloride from the mixture thereof.

Milnacipran base is dissolved in a C₁₋₃ alkanol, preferably in isopropyl alcohol. The dissolution process may also be accompanied by stirring and/or heating to effect complete dissolution. The solution so obtained is treated with hydrochloric acid. The hydrochloric acid may be added as an alcoholic solution, such as an isopropyl alcohol solution. The treatment with hydrochloric acid may be carried out by adding the alcoholic solution of hydrochloric acid to the solution of milnacipran in C₁₋₃ alkanol at about 0° C. to about 30° C. The solution so obtained is treated with a C₄₋₁₀ alkane. The C₄₋₁₀ alkane may be selected from the group consisting of pentane, hexane, heptane and iso-octane. The reaction mixture so obtained is stirred to effect complete precipitation of the solid. The solid so obtained is isolated from the mixture by filtration and/or concentration to obtain Form III of milnacipran hydrochloride.

The present invention provides polymorphic Form N of milnacipran hydrochloride having substantially the same XRPD pattern as depicted in FIG. 8 of the accompanied drawing. The XRPD of Form IV shows characteristic at peaks 2θ values 5.99, 11.53, 11.95, 14.36, 17.94, 18.40, 18.69, 21.13, 21.69, 23.0, 23.39, 23.98, 24.26, 24.50, 26.33, 30.09, 31.70, 33.47, and 33.98±0.2θ. Form IV is further characterized by peaks at 2θ values 4.23, 4.98, 5.40, 5.99, 10.78, 11.53, 11.95, 14.36, 15.55, 16.58, 17.04, 17.94, 18.40, 18.69, 19.26, 20.44, 21.13, 21.69, 22.16, 23.0, 23.39, 23.98, 24.26, 24.50, 25.95, 26.33, 26.94, 27.32, 27.98, 28.71, 29.53, 30.09, 31.18, 31.70, 32.28, 33.47, 33.98, 34.83, 35.43 and 35.65±0.2θ.

Another aspect of the present invention provides a process for the preparation of the polymorphic. Form IV of milnacipran hydrochloride, which comprises
a) dissolving milnacipran base in a C₁₋₃ alkanol,
b) treating the solution obtained in step a) with hydrochloric acid,
c) treating the solution obtained in step b) with an aliphatic ester solvent, and

Milnacipran base is dissolved in a C₁₋₃ alkanol, preferably in isopropyl alcohol. The dissolution process may also be accompanied by stirring and/or heating to effect complete dissolution. The solution so obtained is treated with hydrochloric acid. The hydrochloric acid may be added as an alcoholic solution, such as an isopropyl alcohol solution. The treatment with hydrochloric acid may be carried out by adding the alcoholic solution of hydrochloric acid to the solution of milnacipran in C₁₋₃ alkanol at about 0° C. to about 30° C. The solution so obtained is treated with an aliphatic ester solvent. The aliphatic ester solvent may be ethyl acetate. The reaction mixture so obtained is stirred to effect complete precipitation of the solid. The solid so obtained is isolated from the mixture by filtration and/or concentration to obtain Form IV of milnacipran hydrochloride.

Yet another aspect of the present invention provides a process for the preparation of the polymorphic Form IV of milnacipran hydrochloride, which comprises
a) treating milnacipran base with an aliphatic ester solvent,
b) treating the reaction mixture obtained in step a) with an alcoholic solution of hydrochloric acid, and
c) isolating Form IV of milnacipran hydrochloride from the mixture thereof.

Milnacipran base is treated with an aliphatic ester solvent. The aliphatic ester solvent maybe ethyl acetate. The reaction mixture so obtained is further treated with hydrochloric acid. The hydrochloric acid may be an alcoholic solution, such as an isopropyl alcohol solution of hydrochloric acid is used. The reaction mixture so obtained is stirred to effect complete precipitation of the solid. The stirring is carried out at a temperature of about 20° C. or less. The solid so obtained is isolated from the reaction mixture by filtration and/or concentration to obtain Form IV of milnacipran hydrochloride.

A further aspect of the present invention provides a polymorphic Form V of milnacipran hydrochloride having substantially the same XRPD pattern as depicted in FIG. 9 of the accompanied drawing. The XRPD of Form V shows characteristic peaks at 2θ values 5.96, 7.74, 11.44, 11.92, 14.33, 15.52, 16.55, 17.02, 17.92, 18.37, 18.67, 19.23, 20.44, 21.09, 21.67, 22.14, 22.96, 23.96, 24.24, 25.08, 25.93, 26.64, 26.91, 27.29, 27.97, 28.64, 29.70, 30.06, 31.15, 31.64, 32.25, 34.80 and 35.42±0.2. Form V is further characterized by the absence of peaks between 2θ values of 12.00 and 14.00. The polymorphic Form V has substantially the same FUR pattern as depicted in FIG. 10 of the accompanied drawing. The polymorphic Form V has substantially the same DSC thermogram as depicted in FIG. 11 of the accompanied drawing. The DSC thermogram shows three characteristic endothermic peaks between about 90° and about 100° C., about 125° and about 145° C. and, about 160° and about 175° C.

Yet another aspect of the present invention provides a process for the preparation of the polymorphic Form V of milnacipran hydrochloride, which comprises
a) treating milnacipran hydrochloride with a ketone solvent,
b) heating the reaction mixture obtained in step a) to a temperature of about 60° C. or more,
c) cooling the reaction mixture obtained in step b) to a temperature of about 35° C. or less, and
d) isolating Form V of milnacipran hydrochloride from the mixture thereof.

Milnacipran hydrochloride is treated with a ketone solvent and heated to a temperature of about 60° C. or above. The ketone solvent may be methyl isobutyl ketone or methyl ethyl ketone. The reaction mixture so obtained is subsequently cooled to a temperature of about 35° C. or less. The solid so obtained is isolated from the mixture by filtration and/or concentration to obtain Form V of milnacipran hydrochloride.

Milnacipran base or hydrochloride salt present in any solid form can be used as a starting material for all of the processes of this invention. The starting milnacipran base or hydrochloride salt can be prepared by following the methods provided in U.S. Pat. No. 4,478,836, EP Patent No. 0 200 638 B1, or Japanese Patent No. 2006-008569 A2.

The present invention provides a pharmaceutical composition comprising polymorphic Form I of milnacipran hydrochloride and excipients/diluents.

The present invention also provides a pharmaceutical composition comprising polymorphic Form II of milnacipran hydrochloride and excipients/diluents.

Another aspect of the present invention provides a pharmaceutical composition comprising polymorphic Form III of milnacipran hydrochloride and excipients/diluents.

A further aspect of the present invention provides a pharmaceutical composition comprising polymorphic Form N of milnacipran hydrochloride and excipients/diluents.

An aspect of the present invention provides a pharmaceutical composition comprising polymorphic Form V of milnacipran hydrochloride and excipients/diluents.

Another aspect of the present invention provides a method of treating depression and/or chronic pain conditions such as Fibromyalgia and Lupus, which comprises of administering to a mammal in need thereof a therapeutically effective amount of polymorphic Form I of milnacipran hydrochloride.

A further aspect of the present invention provides a method of treating depression and/or chronic pain conditions such as Fibromyalgia and Lupus, which comprises of administering to a mammal in need thereof a therapeutically effective amount of polymorphic Form II of milnacipran hydrochloride.

Yet another aspect of the present invention provides a method of treating depression and/or chronic pain conditions such as Fibromyalgia and Lupus, which comprises of administering to a mammal in need thereof a therapeutically effective amount of polymorphic Form III of milnacipran hydrochloride.

The present invention also provides a method of treating depression and/or chronic pain conditions such as Fibromyalgia and Lupus, which comprises of administering to a mammal in need thereof a therapeutically effective amount of polymorphic Form IV of milnacipran hydrochloride.

The present invention further provides a method of treating depression and/or chronic pain conditions such as Fibromyalgia and Lupus, which comprises of administering to a mammal in need thereof a therapeutically effective amount of polymorphic Form V of milnacipran hydrochloride.

### BRIEF DESCRIPTION OF THE DRAWINGS

FIG. 1 depicts the XRPD of Form I of milnacipran hydrochloride.

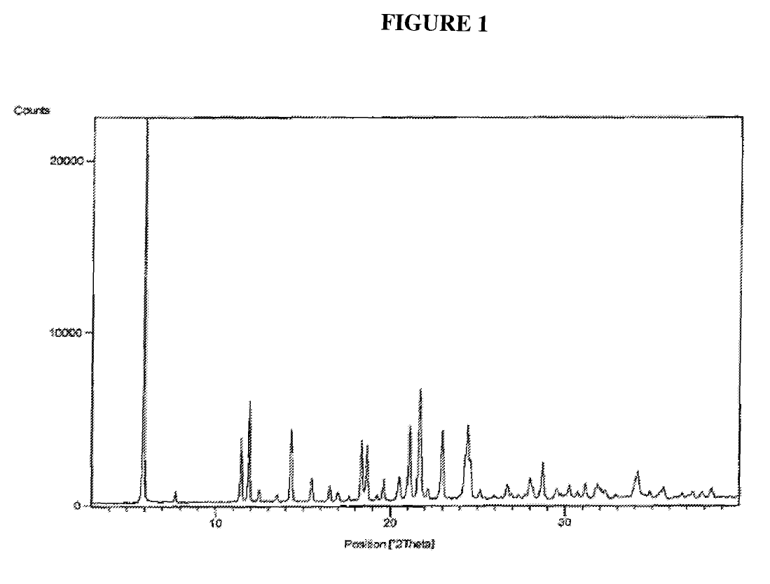

FIG. 2 depicts the FTIR of Form I of milnacipran hydrochloride.

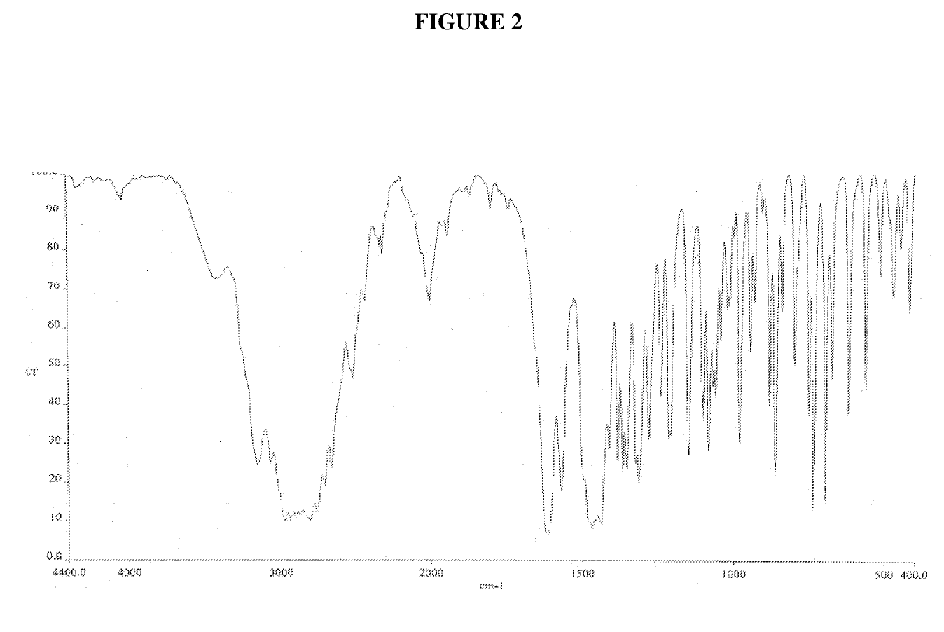

FIG. 3 depicts the DSC of Form I of milnacipran hydrochloride.

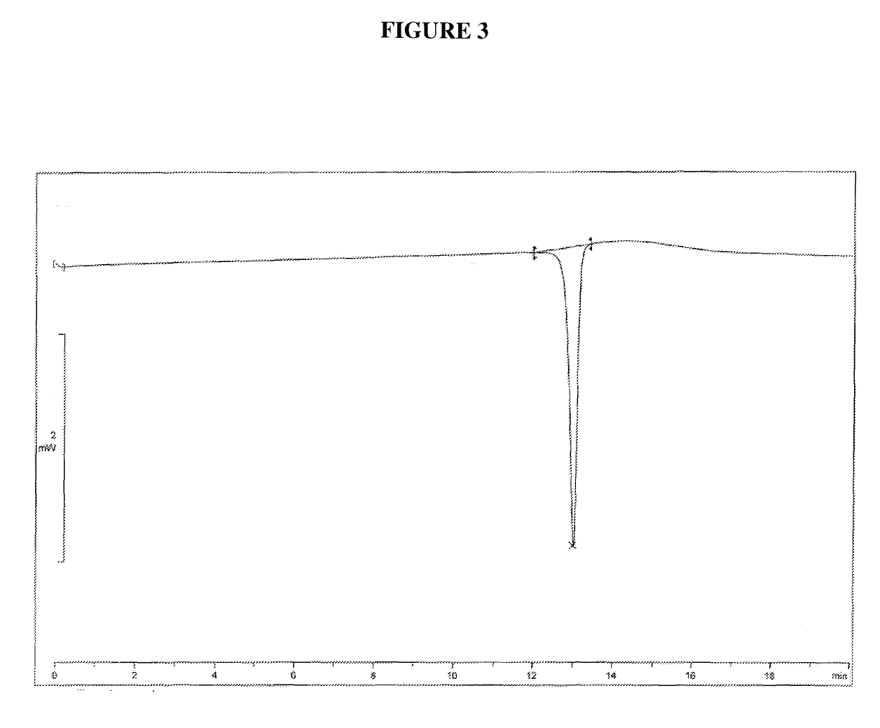

FIG. 4 depicts the XRPD of Form II of milnacipran hydrochloride.

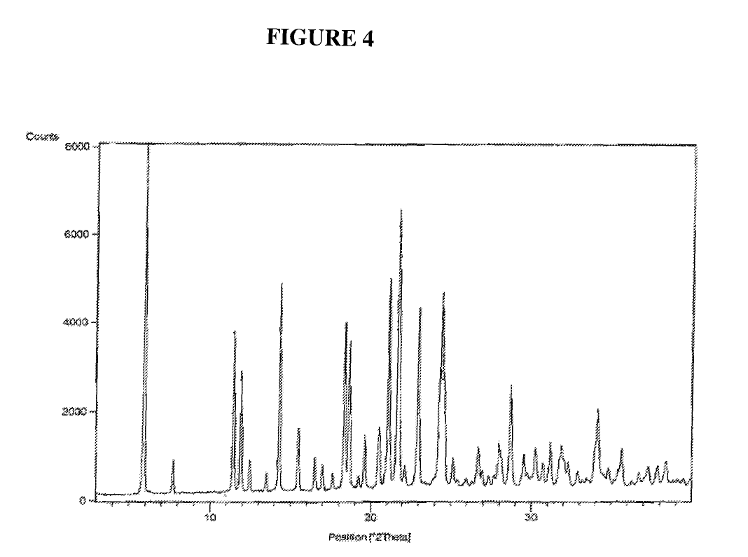

FIG. 5 depicts the FTIR of Form II of milnacipran hydrochloride.

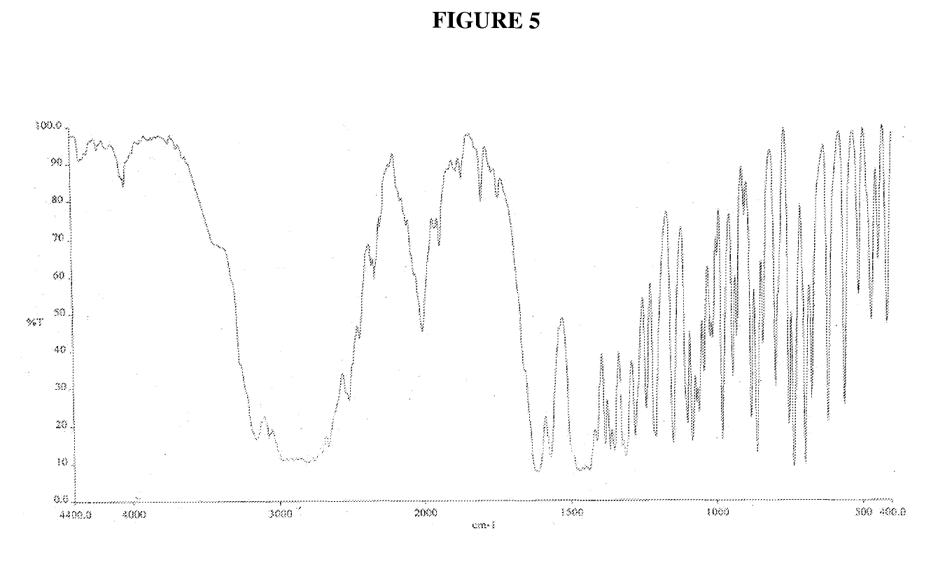

FIG. 6 depicts the DSC of Form II of milnacipran hydrochloride.

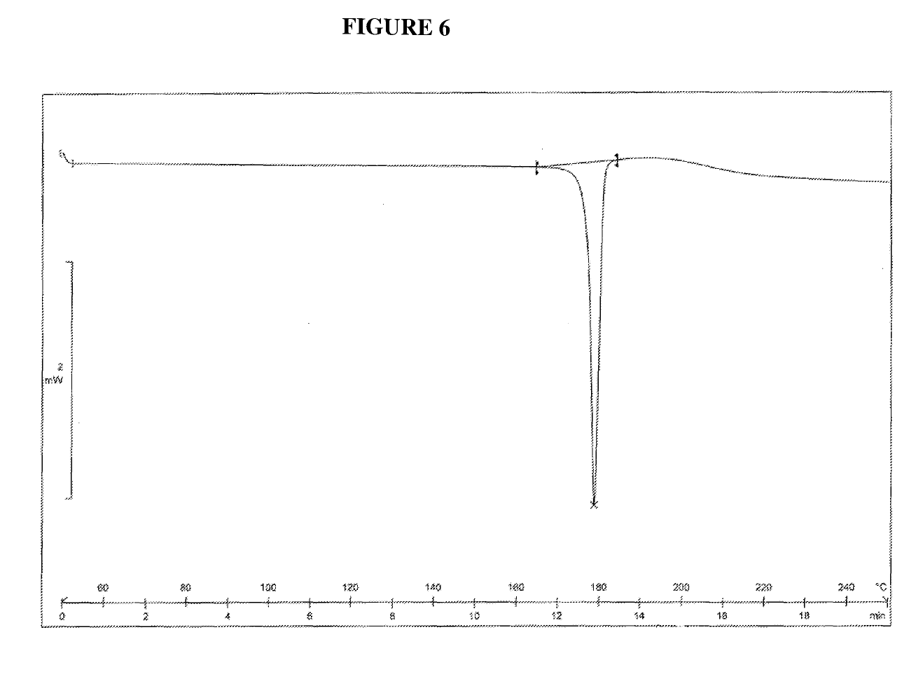

FIG. 7 depicts the XRPD of Form III of milnacipran hydrochloride.

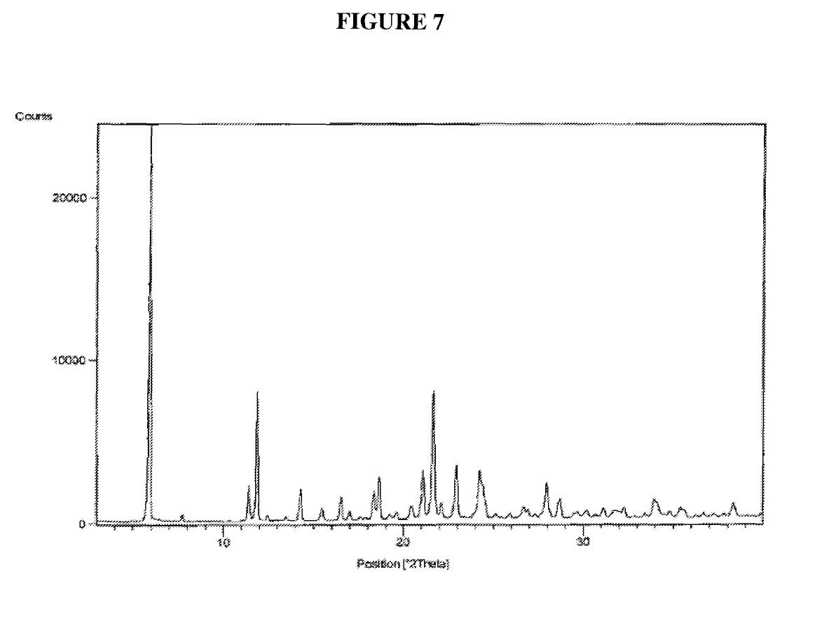

FIG. 8 depicts the XRPD of Form IV of milnacipran hydrochloride.

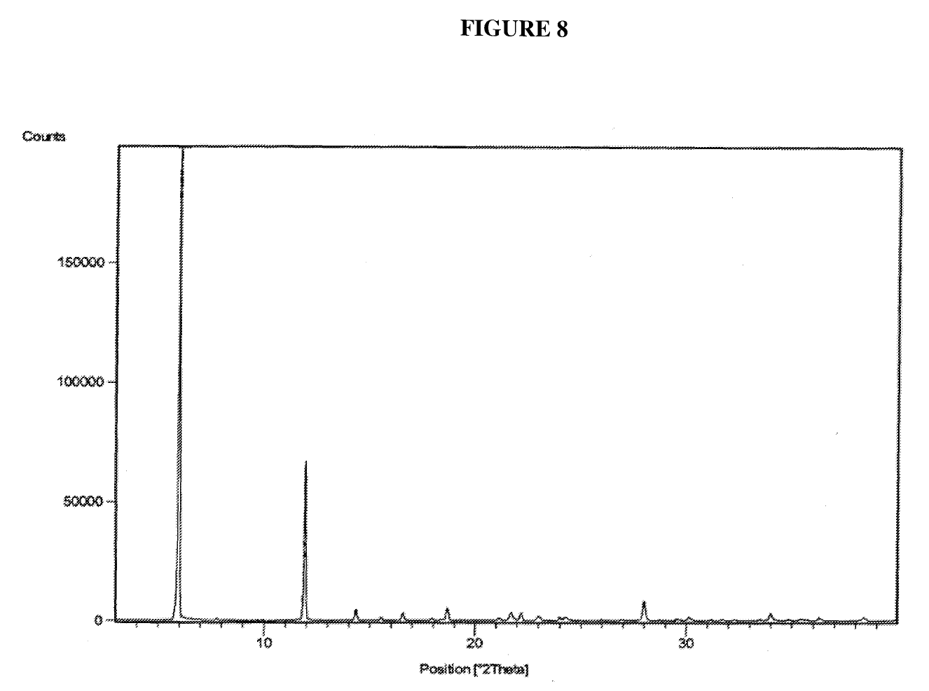

FIG. 9 depicts the XRPD of Form V of milnacipran hydrochloride.

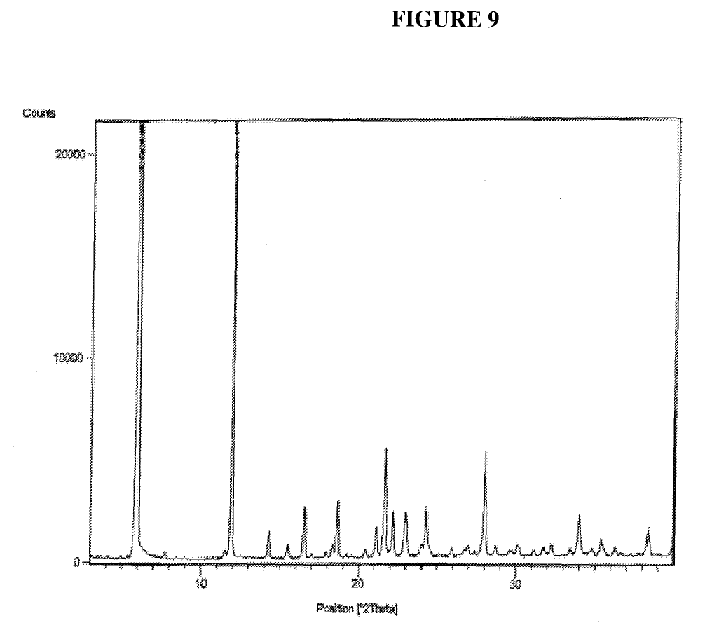

FIG. 10 depicts the FTIR of Form V of milnacipran hydrochloride.

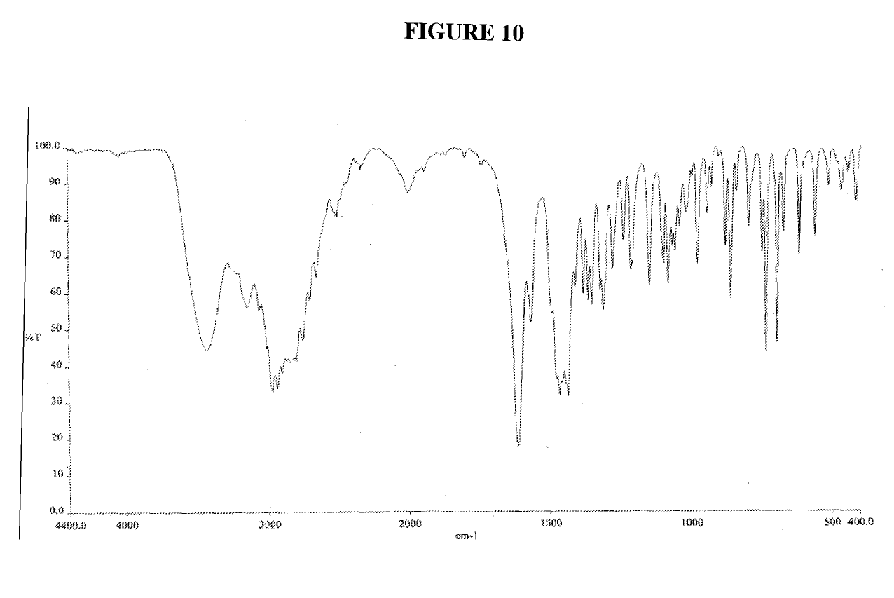

FIG. 11 depicts the DSC of Form V of milnacipran hydrochloride.

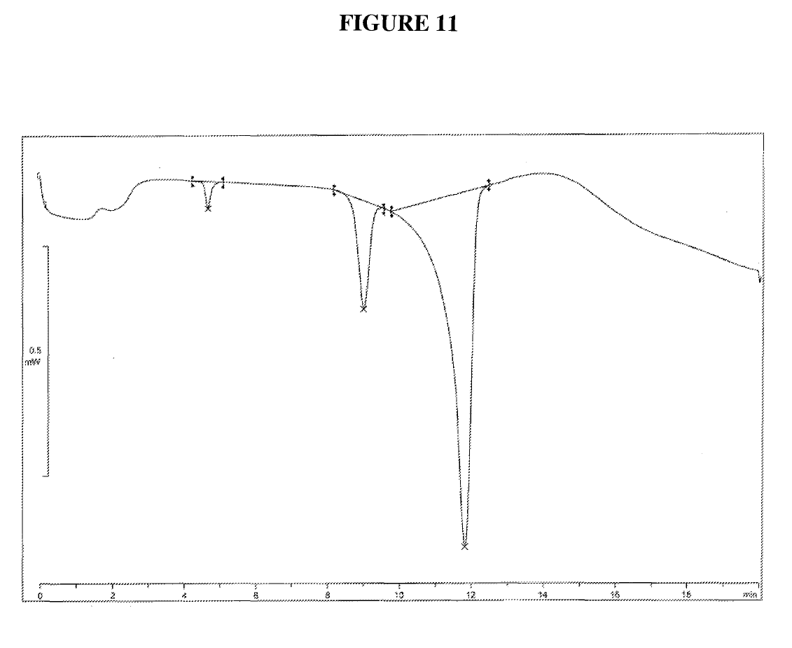

Powder XRD of the samples were determined by using X-Ray Difractometer, Rigaku Corporation, RU-H3R, Goniometer CN2155A3, X-Ray tube with Cu target anode, Divergence slits 1 0, Receiving slit 0.15 mm, Scatter slit 1°, Power: 40 KV, 100 mA, Scanning speed: 2 deg/min step: 0.02 deg, Wave length: 1.5406 A.

FTIR of the samples were determined by using Instrument: Perkin Elmer, 16 PC, SCAN: 16 scans, 4.0 cm⁻¹, according to the USP 25, general test methods page 1920, infrared absorption spectrum by potassium bromide pellet method.

DSC thermograms were recorded using DSC821 e, Mettler Toledo, Sample weight: 3-5 mg, Temperature range: 50-250° C., Heating rate: 10° C./min, Nitrogen 50.0 mL/min, Number of holes in the crucible: 1

While the present invention has been described in terms of its specific embodiments, certain modifications and equivalents will be apparent to those skilled in the art and are intended to be included within the scope of the present invention.

### EXAMPLES

### Example 1

### Preparation of Milnacipran Hydrochloride

### Method A:

(Z)-1-phenyl-1-diethylaminocarbonyl-2-phthalimidomethyl cyclopropane (50 g) was added into denaturated spirit (250 ml) at 25° to 30° C. The reaction mixture was heated to 60° C. and hydrazine hydrate (7.35 g) was added. The reaction mixture was subsequently heated to reflux at 80° C. and stirred at reflux temperature for 3 h. The denaturated spirit was recovered at 50° to 55° C. under reduced pressure, followed by the addition of deionised water (2×50 ml) and the recovery of denaturated spirit was continued under reduced pressure. The mixture obtained was cooled to 10° to 15° C., stirred for 30 minutes at 10° to 15° C. and filtered. The solid was washed with deionised water (10° to 15° C., 2×10 ml) and subjected to suction under vacuum. The wet material obtained was suspended in deionised water (450 ml), cooled to 10° to 15° C. and acidified with concentrated hydrochloric acid (11.4 ml) to about pH 2. The reaction mixture was filtered on Celite bed and washed with deionised water (10° to 15° C., 2×50 ml). The mother liquor and washings were combined and basified with aqueous sodium hydroxide solution (about 25% w/v) to attain a pH of about 12 at 10° to 15° C. The reaction mixture was extracted with dichloromethane (3×50 ml) and combined organic layers were stirred with activated carbon (1.0 g at 25° to 30° C. for 1 h). The contents were filtered over Celite bed and washed with dichloromethane (2×10 ml). The mother liquor and washings were combined and the solvent was removed under reduced pressure to obtain a residue. Isopropyl alcohol (5 ml) was added to the residue, followed by the addition of ethyl acetate (250 ml) at 25° to 30° C. Isopropyl alcohol hydrochloride (15 g) was added to the reaction mixture in 1 h at 25° to 40° C. to obtain a pH of about 2. The reaction mixture was heated to reflux (about 80° C.) and stirred for 2 h. The slurry obtained was cooled to 10° to 15° C. and stirred for 1 h at 10° to 15° C. The slurry was filtered, washed with ethyl acetate (2×30 ml, 10° to 15° C.), and further washed with ethyl acetate (30 ml, 10-15° C.) at faster rate. The wet solid (28.5 g) obtained was dried at 50° to 55° C. under vacuum to obtain the title compound.

Yield: 21.2 g

### Method B:

(Z)-1-phenyl-1-diethylaminocarbonyl-2-phthalimidomethyl cyclopropane (2.5 Kg) was added into denaturated spirit (12.5 L) stirred at about 25° C. The reaction mixture was further stirred for 10 minutes, followed by the addition of hydrazine hydrate (0.375 Kg). The reaction mixture was heated at reflux temperature (80° C.) for 3 h. The denaturated spirit was removed at 50° to 55° C. under reduced pressure, followed by the addition of deionised water (2.5 L) and the recovery of denaturated spirit was continued at 50° to 55° C. under reduced pressure. The mixture obtained was cooled to 10° to 15° C., stirred for 30 minutes at 10° to 15° C. and filtered. The solid obtained was washed with cooled deionised water (10° to 15° C., 2×0.5 L) and subjected to suction under vacuum. The wet material was added to deionised water (7.5 L) and cooled to 10° to 15° C. Concentrated hydrochloric acid (about 0.57 L) was added to the reaction mixture to obtain a pH of about 2, and stirred for 15 minutes at 10° to 15° C. The mixture obtained was filtered over Celite bed and washed with cooled deionised water (10° to 15° C., 2×2.5 L) The filtrate and washings were combined and aqueous sodium hydroxide solution (prepared by dissolving sodium hydroxide (0.25 Kg) in deionised water (1 L) and cooling the solution to 10° C.) was added at 10° to 15° C. to obtain a pH of about 12. Dichloromethane (2.5 L) was added to the reaction mixture at 10° to 15° C. and stirred for 10 minutes at 10° to 15° C. The reaction mixture was filtered through Celite bed, washed with dichloromethane (2×0.5 L) and the layers were allowed to settle. The organic layer (OL-1) and the aqueous layer (AL-1) were separated. Dichloromethane (2.5 L) was added to the aqueous layer (AL-1), stirred for 10 minutes at 10° to 15° C. and the layers were allowed to settle. The organic layer (OL-2) and the aqueous layer (AL-2) were separated.

Dichloromethane (2.5 L) was added to the aqueous layer (AL-2), stirred for 10 minutes at 10° to 15° C. and the layers were allowed to settle. The organic layer (OL-3) and the aqueous layer (AL-3) were separated and the aqueous layer (AL-3) was discarded. The organic layers (OL-1+OL-2+OL-3) were combined and activated carbon (0.05 Kg) was added to the combined organic layer. The reaction mixture was stirred for 1 h at 25° to 30° C. The mixture obtained was filtered through Celite bed, washed with dichloromethane (2×0.5 L). The filtrate and the washings were combined. Dichloromethane was recovered initially at atmospheric pressure up to 50% of volume and subsequently under reduced pressure at 35° to 40° C. Ethyl acetate (2.5 L) was added to the residue and stirred for 15 minutes. Ethyl acetate was recovered at 40° to 45° C. under reduced pressure. Ethyl acetate (12.5 L) was added to the residue, and stirred at 25° to 30° C. Isopropyl alcohol hydrochloride (0.8 Kg) was added to the reaction mixture in 1 h at 25° to 40° C. and stirred for 30 minutes at 25° to 30° C. The reaction mixture was cooled to 10° to 15° C. and stirred for 1 hr at 10° to 15° C. The reaction mixture was filtered, washed with pre-cooled ethyl acetate (2×1.5 L, 10° to 15° C.) and further washed with ethyl acetate (1.5 L, 10-15° C.) at faster rate. The wet solid was dried at 45° to 55° C. under vacuum to obtain the title compound.

Yield: 1.18 kg

HPLC Purity: 99.98%

### Example 2

### Preparation of Form I of Milnacipran Hydrochloride

Milnacipran hydrochloride (5 g) obtained from Example 1 (B) was dissolved in methanol (50 ml) at 25° to 30° C. to obtain a clear solution. Methanol was recovered under vacuum to obtain a solid. The solid was further dried under vacuum to obtain the title compound.

Yield: 4.3 g

### Example 3

### Preparation of Form I of Milnacipran Hydrochloride

Milnacipran hydrochloride (5 g) obtained from Example 1 (B) was dissolved in 1,4-dioxane (350 ml) at 80° to 85° C. to obtain a clear solution. The solution was cooled to 25° to 30° C. The reaction mixture was filtered and washed with 1,4-dioxane (10 ml). The solid was dried under vacuum to obtain the title compound.

Yield: 2.5 g

### Example 4

### Preparation of Form I of Milnacipran Hydrochloride

Milnacipran hydrochloride (5 g) obtained from Example 1 (B) was suspended in toluene (50 ml). The mixture was refluxed at 110° C. for 4 h. The slurry was cooled to 25° to 30° C. and stirred for 1 h. The reaction mixture was filtered and washed with toluene (2×10 ml). The solid was dried under vacuum to obtain the title compound.

Yield: 4.3 g

### Example 5

### Preparation of Form I of Milnacipran Hydrochloride

Milnacipran hydrochloride (50 g) obtained from Example 1 (B) was added to ethyl acetate (250 ml) and stirred at 25° to 30° C. Isopropyl alcohol (5 ml) was added to the reaction mixture, followed by the addition of isopropyl alcohol hydrochloride (3 ml) to attain the pH of about 2 in 15 minutes at 25° to 30° C. The slurry was heated to reflux temperature (about 80° C.) and stirred under reflux for 2 h. The slurry was cooled to 10° to 15° C. and stirred for 1 h at 10° to 15° C. The mixture was filtered and washed with ethyl acetate (2×30 ml, 10° to 15° C.) followed by running wash with ethyl acetate (30 ml, 10° to 15° C.). The wet solid (61 g) was dried at 50° to 55° C. under vacuum to obtain the title compound.

Yield: 48.9 g

### Example 6

### Preparation of Form I of Milnacipran Hydrochloride

Milnacipran hydrochloride (5 g) was dissolved in methanol (5 ml) to obtain a clear solution. The solution so obtained was added to stirred diisopropyl ether (200 ml) at 25° to 30° C. in about 20 minutes. The mixture was stirred for about 1 h at 25° to 30° C., filtered and washed with diisopropyl ether (2×10 ml). The solid obtained was dried under vacuum to obtain the title compound.

Yield: 4.5 g

### Example 7

### Preparation of Form Ii of Milnacipran Hydrochloride

Milnacipran Hydrochloride (20 g) was dissolved in deionised water (200 ml) at 25° to 30° C. to obtain a clear solution. The solution so obtained was spray dried in spray dryer (Model: Lab plant SD-05; Carrier gas: compressed air; Atomizer type: pressure/spray nozzle; Feed rate: 3 ml/minute; Inlet temperature: 95° C.). The solid obtained was dried to get the title compound.

Yield: 8.5 g

### Example 8

### Preparation of Form Iii of Milnacipran Hydrochloride

Milnacipran base (10 g) was dissolved in isopropyl alcohol (10 ml) by stirring. Isopropyl alcohol hydrochloride (10 ml) was added into said solution at 25° to 30° C. followed by stirring for 5 minutes. Hexane (200 ml) was subsequently added into said solution at 20° to 25° C. The reaction mixture was stirred for half an hour at 20° to 25° C. and filtered. The solid was washed with hexane (20 ml) and dried under vacuum over night at 40° to 45° C. to obtain the title compound.

Yield: 9.3 g

### Example 9

### Preparation of Form Iv of Milnacipran Hydrochloride

Milnacipran base (10 g) was dissolved in isopropyl alcohol (10 ml) by stirring. Isopropyl alcohol hydrochloride (10 ml) was added into said solution at 20° to 25° C. and stirred for 10 minutes. Ethyl acetate (250 ml) was subsequently added into said solution and stirred for 1 h at 20° to 25° C. The solid obtained was filtered and washed with ethyl acetate (2×40 ml). The wet material (9.2 g) was dried under suction and further dried overnight at 45° to 50° C. under vacuum to obtain the title compound.

Yield: 8.2 g

### Example 10

### Preparation of Form Iv of Milnacipran Hydrochloride

(Z)-1-phenyl-1-diethylaminocarbonyl-2-phthalimidomethyl cyclopropane (50 g) was added into denaturated spirit (250 ml) at about 25° C., followed by the addition of hydrazine hydrate (7.50 g). The reaction mixture was heated to reflux at 80° C. and stirred at reflux temperature for 3 h. The denaturated spirit was recovered at 50° to 55° C. under reduced pressure. The deionised water (2×50 ml) was added to the reaction mixture and the recovery of denaturated spirit was continued under reduced pressure. The reaction mixture was cooled to 10° to 15° C., stirred for 30 minutes at 10° to 15° C. and filtered. The solid was washed with deionised water (10° to 15° C., 2×10 ml) and subjected to suction under vacuum. The wet material obtained was suspended in deionised water (150 ml), cooled to 10° to 15° C. and acidified with concentrated hydrochloric acid (11.2 ml) to obtain a pH of about 2. The reaction mixture was filtered on Celite bed and washed with deionised water (10° to 15° C., 2×50 ml). The mother liquor and the washings were combined and basified with aqueous sodium hydroxide solution (about 25% w/v) to obtain a pH of about 12 at 10° to 15° C. The mixture was extracted with dichloromethane (3×50 ml) and the combined organic layers were stirred with activated carbon (1.0 g, 25° to 30° C., 1 h). The reaction mixture was filtered over Celite bed and washed with dichloromethane (2×10 ml). The mother liquor and the washings were combined and the solvent was recovered under reduced pressure to obtain an oily residue. Ethyl acetate (250 ml) was added to the residue and stirred at 25° to 30° C. Isopropyl alcohol hydrochloride (15 g) was added to the reaction mixture in 1 h at 25° to 40° C. The reaction mixture was stirred for 30 minutes at 25° to 30° C., cooled to 10° to 15° C. and stirred for 1 h at 10° to 15° C. The reaction mixture was filtered and the slurry was washed with ethyl acetate (2×30 ml, 10° to 15° C.) and running wash was carried out with ethyl acetate (30 ml, 10° to 15° C.). The wet solid was dried at 45° to 55° C. under vacuum to obtain the title compound.

Yield: 21.7 g

### Example 11

### Preparation of Form V of Milnacipran Hydrochloride

Milnacipran hydrochloride (5 g) obtained from Example 1 (B) was suspended in methyl isobutyl ketone (50 ml). The reaction mixture was refluxed at 118° C. for 4 h. The slurry was cooled to 25° to 30° C. and stirred for 1 h. The reaction mixture was filtered and washed with methyl isobutyl ketone (2×10 ml). The solid was dried under vacuum to obtain the title compound.

Yield: 4.3 g

We claim:

1. Polymorphic Form I of milnacipran hydrochloride having an XRPD pattern, wherein peaks are obtained at 2θ values 5.97, 7.75, 11.46, 11.93, 12.48, 13.52, 14.32, 15.52, 16.56, 17.03, 17.63, 18.37, 18.67, 19.26, 19.63, 20.54, 21.11, 21.68, 22.15, 22.96, 24.24, 24.38, 24.58, 25.12, 25.91, 26.69, 26.93, 27.31, 27.98, 28.70, 29.51, 30.25, 30.70, 31.15, 31.84, 32.29, 32.87, 34.13, 34.83 and 35.64±0.2θ.

2. Polymorphic Form I of milnacipran hydrochloride having a DSC pattern, wherein one characteristic endothermic peak is obtained between about 177° and about 179° C.

3. A process for the preparation of polymorphic Form I of milnacipran hydrochloride, which comprises,
a) dissolving milnacipran hydrochloride in a C₁₋₃ alkanol,
b) treating the solution obtained in step a) with an ether solvent, and
c) isolating Form I of milnacipran hydrochloride from the mixture thereof.

4. A process for the preparation of polymorphic Form I of milnacipran hydrochloride, which comprises
a) dissolving milnacipran hydrochloride in a C₁₋₃ alkanol,
b) partially or completely removing the solvent from the solution obtained in step a), and
c) isolating Form I of milnacipran hydrochloride from the mixture thereof.

5. A process for the preparation of polymorphic Form I of milnacipran hydrochloride, which comprises,
a) dissolving milnacipran hydrochloride in a water miscible ether at a temperature of about 50° C. or more,
b) cooling the solution obtained in step a) to a temperature of about 35° C. or less, and
c) isolating Form I of milnacipran hydrochloride from the mixture thereof.

6. A process for the preparation of polymorphic Form I of milnacipran hydrochloride, which comprises
a) treating milnacipran hydrochloride with an aromatic hydrocarbon solvent,
b) heating the reaction mixture obtained in step a) to a temperature of about 60° C. or more,
c) cooling the reaction mixture obtained in step b) to a temperature of about 35° C. or less, and
d) isolating Form I of milnacipran hydrochloride from the mixture thereof.

7. A process for the preparation of polymorphic Form I of milnacipran hydrochloride, which comprises
a) treating milnacipran hydrochloride with an aliphatic ester solvent,
b) treating the mixture obtained in step a) with an acid, and
c) isolating Form I of milnacipran hydrochloride from the mixture thereof.

8. The process of claim 7 wherein a C₁₋₃ alkanol is additionally used with the aliphatic ester in step a).

9. Polymorphic Form II of milnacipran hydrochloride having an XRPD pattern, wherein peaks are obtained at 2θ values 5.95, 11.45, 11.91, 14.32, 18.37, 18.66, 21.11, 21.67, 22.96, 24.23, 24.38, 24.57, 25.39 and 27.67±0.2θ.

10. Polymorphic Form II of milnacipran hydrochloride having a DSC pattern, wherein one characteristic endothermic peak is obtained between about 175° and about 177° C.

11. A process for the preparation of polymorphic Form II of milnacipran hydrochloride, which comprises
a) dissolving milnacipran hydrochloride in an aqueous solvent,
b) spray drying the solution obtained in step a) in a spray dryer, and
c) isolating Form II of milnacipran hydrochloride from the spray dryer.

12. Polymorphic Form III of milnacipran hydrochloride having an XRPD pattern, wherein peaks are obtained at 2θ values 5.94, 11.44, 11.90, 14.31, 18.36, 18.65, 21.10, 21.66, 22.95, 24.22, 24.57, 29.70, 31.68, 33.42, 33.93, and 35.39±0.2θ.

13. A process for the preparation of polymorphic Form III of milnacipran hydrochloride, which comprises
a) dissolving milnacipran base in a C₁₋₃ alkanol,
b) treating the solution obtained in step a) with hydrochloric acid,
c) treating the solution obtained in step b) with a C₄₋₁₀ alkane, and
d) isolating Form III of milnacipran hydrochloride from the mixture thereof.

14. Polymorphic Form IV of milnacipran hydrochloride having an XRPD pattern, wherein peaks are obtained at 2θ values 5.99, 10.78, 11.53, 11.95, 14.36, 17.94, 18.40, 18.69, 21.13, 21.69, 23.0, 23.39, 23.98, 24.26, 24.50, 26.33, 30.09, 31.70, 33.47, and 33.98±0.2θ.

15. A process for the preparation of polymorphic Form IV of milnacipran hydrochloride, which comprises
a) dissolving milnacipran base in a C₁₋₃ alkanol,
b) treating the solution obtained in step a) with hydrochloric acid,
c) treating the solution obtained in step b) with an aliphatic ester solvent, and
d) isolating Form IV of milnacipran hydrochloride from the mixture thereof.

16. A process for the preparation of polymorphic Form IV of milnacipran hydrochloride, which comprises
a) treating milnacipran base with an aliphatic ester solvent,
b) treating the reaction mixture obtained in step a) with an alcoholic solution of hydrochloric acid, and
c) isolating Form IV of milnacipran hydrochloride from the mixture thereof.

17. Polymorphic Form V of milnacipran hydrochloride having an XRPD wherein peaks are obtained at 2θ values 5.96, 7.74, 11.44, 11.92, 14.33, 15.52, 16.55, 17.02, 17.92, 18.37, 18.67, 19.23, 20.44, 21.09, 21.67, 22.14, 22.96, 23.96, 24.24, 25.08, 25.93, 26.64, 26.91, 27.29, 27.97, 28.64, 29.70, 30.06, 31.15, 31.64, 32.25, 34.80 and 35.42±0.2θ.

18. Polymorphic Form V of milnacipran hydrochloride having a DSC thermogram wherein three endothermic peaks are obtained between about 90° and about 100° C., about 125° and about 145° C., and about 160° and about 175° C.

19. A process for the preparation of polymorphic Form V of milnacipran hydrochloride, which comprises
a) treating milnacipran hydrochloride with a ketone solvent,
b) heating the reaction mixture obtained in step a) to a temperature of about 60° C. or more,
c) cooling the reaction mixture obtained in step b) to a temperature of about 35° C. or less, and
d) isolating Form V of milnacipran hydrochloride from the mixture thereof.
